(19) United States
(12) Patent Application Publication
(10) Pub. No.: US 2006/0004295 A1
(43) Pub. Date: Jan. 5, 2006

(54) Electrode connectivity determination system

(76) Inventor: Randolph Prydekker, Wilmot, NH (US)

Correspondence Address: JACK SCHWARTZ & ASSOCIATES, 1350 BROADWAY, SUITE 1510, NEW YORK, NY, 10018, US

(21) Appl. No.: 11/168,499

(22) Filed: Jun. 28, 2005

Related U.S. Application Data
(60) Provisional application No. 60/583,812, filed on Jun. 29, 2004.

(51) Int. Cl. A61B 5/04

(52) U.S. Cl. 600/509

(57) ABSTRACT

An electrode connectivity determination system is coupled to a plurality of patient attachable electrodes. Such a system includes a comparator for performing a comparison of a low frequency differential signal derived from at least two of the patient attachable electrodes with a predetermined threshold signal and a decision processor for generating a signal indicating an electrode signal is at least potentially degraded in response to said first comparison.

### CROSS REFERNCE TO RELATED APPLICATIONS

This is a non-provisional application of provisional application Ser. No. 60/583,812 By Randolph Predekker filed on Jun. 29, 2004.

### FIELD OF THE INVENTION

The present invention relates to a system for determining the connectivity of electrodes, and in particular for determining the connectivity of electrodes to a patient in a medical monitoring and/or treatment device.

### BACKGROUND OF THE INVENTION

Patient monitoring and/or treatment systems often use patient attachable electrodes. As one example, ECG systems are well known and provide information about the physiological status of a patient's heart to a physician. So-called conventional 12 lead ECG systems exist (both wired and wireless) which provide twelve waveforms, called ECG leads or lead signals, to a physician. To provide such a 12 lead ECG, ten patient attachable electrodes are attached to predefined locations on a patient's body, and the signals from these patient attachable electrodes are processed to provide the twelve ECG lead signals. The respective ECG lead signals are based on differential signals derived from two or more of the signals from patient attachable electrodes, in a known manner. These ten patient attachable electrodes include four patient attachable electrodes which provide signals that are processed to generate six limb ECG lead signals, and six patient attachable electrodes which provide signals that are processed to provide six precordial or chest ECG lead signals. It is also possible that a subset or reduced number of patient attachable electrodes may be used, compared to a conventional 12 ECG lead signal, for example, if surgery or injury prevents attachment of a patient attachable electrode at a predefined location. For example, a six ECG lead system may be generated by attaching six electrodes. It is also possible that a superset or additional number of patient attachable electrodes may be used, compared to a conventional 12 ECG lead system to provide additional data to a physician about a patient cardiac function.

A patient attachable electrode signal may become at least potentially degraded if the electrical contact between the patient attachable electrode and a patient body is impaired. Such an impaired electrical contact may occur if (a) the patient attachable electrode is detached from said patient body or (b) the electrical resistivity between the patient attachable electrode and the patient body is significantly increased because of degraded electrical contact. When the signal from a patient attachable electrode becomes degraded, one or more ECG lead signals based on the electrical signal from that patient attachable electrode may be inaccurate.

Existing patient monitoring and/or treatment devices, such as ECG measurement systems, incorporate some form of decision making related to the suitability of ECG lead signals for algorithm processing and display. One known method for detecting this open circuit condition is by using a resistive divider and comparator scheme. In this scheme typically a resistive divider converts the series resistance of skin, patient attachable electrode, wire and processing circuitry to a measurable signal. More specifically, the measurable signal is a low frequency or DC signal. A comparator determines when this measurable signal exceeds a predetermined threshold value. When the threshold value is exceeded the display and processing of an affected ECG lead or leads based on the patient attachable electrode is inhibited. As used herein, a comparator is a circuit which compares a first signal to a second and generates a signal representing the result of the comparison. Typically the signal is a bistate signal having a first state when the first signal is greater than or equal to the second signal and a second state when the first signal is less than the second signal.

FIG. 1 is a diagram illustrating schematically the arrangement of patient attachable electrodes on a patient 102 and a portion of electrical circuitry associated with the electrodes. In FIG. 1, six patient attachable electrodes, right leg RL, left leg LL, right arm RA, left arm LA, first chest C1 and second chest C2, are illustrated by circles. These are representative only and illustrated to simplify the figure. One skilled in the art understands that other patient attachable electrodes (not shown) may be attached to the patient 102 at other predefined locations.

An electric voltage source 104, illustrated in FIG. 1 as a battery, has a first terminal coupled to a reference patient attachable electrode RL and patient attachable electrode terminal E_{RL} via a first resistance R_{SC}, in a common branch. The second terminal of the battery 104 is coupled in common to the other patient attachable electrodes, LL, LA, RA, C1, C2, via respective series connections of pull-down resistors R_{PD}, patient attachable electrode terminals, E_{LL}, E_{LA}, E_{RA}, E_{C1}, E_{C2}, and branch resistors R_{BR} in corresponding non-common branches. The electrode signal terminals E_{RL}, E_{LL}, E_{LA}, E_{RA}, E_{C1}, E_{C2} are coupled to ECG processing and display circuitry (not shown). In the description below, E_{LL}, E_{LA}, E_{RA}, E_{C1}, E_{C2}, are used to designate the patient attachable electrode terminals, and the signals produced at those terminals. One skilled in the art understands from the context whether the signal or terminal is referred to.

In operation, the voltage source 104 produces a voltage REF at the first terminal with respect to the voltage at the second terminal. The reference patient attachable electrode RL is generally maintained at a constant potential, and establishes a reference potential on the patient 102 at the location of the RL patient attachable electrode. The reference patient attachable electrode terminal E_{RL} produces this reference voltage and is coupled to the processing circuitry (not shown) to provide reference and monitoring.

The ECG signal voltages at the non-common patient attachable electrode terminals, E_{LL}, E_{LA}, E_{RA}, E_{C1}, E_{C2}, are measured with respect to the reference voltage REF at terminal E_{RL} and are processed by the ECG processing circuitry (not shown) to produce differential ECG lead signals (I, II, V, V+, etc.) which are displayed for a physician. For example, the I lead signal is generated by subtracting the signal E_{RA} from E_{LA}, the II lead signal is generated by subtracting the signal E_{RA} from E_{LL}, and so forth. The patient attachable electrode signals, E_{LL}, E_{LA}, E_{RA}, E_{C1}, E_{C2}, are also processed to produce respective signals indicating that a patient attachable electrode signal is potentially degraded and that ECG lead signals derived from that patient attachable electrode are also potentially degraded.

FIG. 2 is a more detailed electrical schematic diagram of an equivalent circuit of the electrode connectivity determination system. Elements in FIG. 2 which are the same as those in FIG. 1 are designated by the same reference number and are not described in detail below. The voltage source 104 is represented by a source of reference voltage, which in the illustrated embodiment is 2.5 volts, and a clamp 202. This generates a controlled reference voltage REF which is coupled to the reference electrode signal terminal E_{RL}, and to the RL patient attachable electrode through R_{SC}.

In FIG. 2, the resistivity of the respective patient attachable electrodes, RL, LL, RA, LA, C1, C2, are represented by variable resistances R_{RL}, R_{LL}, R_{RA}, R_{LA}, R_{C1}, and R_{C2}. Ideally, these resistances are relatively low or zero. In the case of a patient attachable electrode separating from a patient, these resistances become an open circuit. In the illustrated embodiment R_{SC} is 157.16 kΩ, R_{BR} is 154 kΩ, and R_{PD} is a relatively high impedance.

In FIG. 2, the patient 102 (FIG. 1) is represented by connecting the patient attachable electrode resistance R_{RL} to the respective patient attachable electrode resistances R_{LL}, R_{RA}, R_{LA}, R_{C1}, R_{C2}. There is a location within the patient at which the voltage E_{X} exists. The combination of R_{SC} in the common branch and the respective R_{BR} and R_{PD} resistances in the non-common branches form corresponding resistor dividers to which the non-common branch patient attachable electrode terminals, E_{LL}, E_{LA}, E_{RA}, E_{C1}, E_{C2}, are coupled. The patient attachable electrode terminals, E_{RL}, E_{LL}, E_{LA}, E_{RA}, E_{C1}, E_{C2}, are also coupled to a plurality of buffer amplifiers and low pass filters 204 which generate low frequency or DC signals E_{RL}(DC), E_{LL}(DC), E_{RA}(DC), E_{LA}(DC), E_{C1}(DC), E_{C2}(DC), representing the low frequency or DC component of the patient attachable electrode signals, E_{RL}, E_{LL}, E_{LA}, E_{RA}, E_{C1}, E_{C2}. The low frequency or DC patient attachable electrode signals, E_{RL}(DC), E_{LL}(DC), E_{RA}(DC), E_{LA}(DC), E_{C1}(DC), E_{C2}(DC), are coupled to a blanking processor 206.

If a patient attachable electrode, RL, LL, RA, LA, C1, C2, detaches from a patient, an open circuit exists in the voltage dividers. For example, if the reference patient attachable electrode RL detaches from the patient, then the voltage dividers have an open circuit. In this case, the DC level of the reference patient attachable electrode terminal E_{RL} remains at the reference voltage REF, and that of the non-common patient attachable electrode terminals are pulled down to ground. If a non-common patient attachable electrode, LL, LA, RA, C1, C2, detaches from the patient then the DC level of that patient attachable electrode signal, E_{LL}(DC), E_{LA}(DC), E_{RA}(DC), E_{C1}(DC), E_{C2}(DC), is pulled down to ground. The DC levels of the other non-common branch patient attachable electrode signals, E_{LL}(DC), E_{LA}(DC), E_{RA}(DC), E_{C1}(DC), E_{C2}(DC), change due to the open circuit caused by the detached patient attachable electrode, LL, LA, RA, C1, C2 but remain close to their typical DC levels.

The blanking processor 206 includes a plurality of comparators 210, having respective first input terminals responsive to corresponding low frequency or DC patient attachable electrode signals Eᵢ(DC), second input terminals coupled to a source of a threshold signal TH, and output terminals coupled to corresponding blanking output terminals Bᵢ. If, as described above, a patient attachable electrode has detached, then one or more of the non-common patient attachable electrode terminals are pulled down to ground. The threshold TH is set to detect this condition. If a patient attachable electrode signal exceeds the threshold, then the corresponding blanking signal is set to a first state indicating that the patient attachable electrode signal is degraded. Otherwise, i.e. the patient attachable electrode signal is within the threshold, the corresponding blanking signal is set to a second state indicating that the patient attachable electrode signal is not degraded. ECG processing circuitry receives the respective blanking signals B_{LL}, B_{RA}, B_{LA}, B_{C1}, B_{C2} and blanks ECG lead signals depending on degraded patient attachable electrode signals. The blanking processor 206 may also include circuitry to determine which ECG lead signals are affected by the respective patient attachable electrode signals, and may generate ECG lead blanking signals generated so that any ECG lead signal which depends on a degraded patient attachable electrode signal is blanked.

This scheme works properly for patient attachable electrodes which detach from a patient. However, there are other situations in which the resistivities of the patient attachable electrodes increase without becoming an open circuit. For example, patient attachable electrodes dry out over time, causing their resistivity to increase. Also, the skin of older patients is relatively dryer than younger patients. This increases the apparent resistivity of the patient attachable electrodes. Increases in resistivity of one or more patient attachable electrode change the low frequency or DC component of patient attachable electrode signals for those patient attachable electrodes.

The divider/comparator scheme, based on a single threshold criteria, as described above, does not accommodate the fact that the DC voltage for a given patient attachable electrode is a function of a variety of different patient attachable electrode resistances and patients. That is, different patient attachable electrodes may have different electrode resistances and different patients have different skin conductivity, which affect the operation of the divider/comparator scheme. As a consequence, the divider/comparator scheme may work adequately in those cases when a ECG lead falls off. But in other circumstances involving different combinations of patient attachable electrode and/or patient skin resistance, decisions determining whether ECG signal data is to be processed and displayed become less reliable. In general, using such a divider/comparator scheme, if a threshold is chosen to have too high a value, excessively noisy data may be displayed and processed, and if too low a threshold value is selected, good data is inhibited from being processed and displayed.

There are several situations which may adversely affect the operation of the single threshold divider/comparator scheme. For example, as patient attachable electrodes are used over a relatively long period of time, they dry out. Referring again to FIG. 2, in this situation, this concurrently increases the resistances, R_{RL}, R_{LL}, R_{LA}, R_{RA}, R_{C1}, R_{C2}, of the patient attachable electrodes, RL, LL, LA, RA, C1, C2. This changes the parameters of the respective voltage dividers and decreases the DC voltages for the non-common patient attachable electrodes. However, the differential amplifiers (not shown) generating the ECG lead signals are still operating within acceptable constraints. Similarly, the resistance R_{RL} of the patient attachable electrode in the common branch, or the resistance, R_{LL}, R_{LA}, R_{RA}, R_{C1}, R_{C2}, of one of the non-common patient attachable electrodes may increase, due, for example, to impaired electrical contact with the patient and/or dry skin on a patient leading to similar operations as described above. The prior art system would blank the non-common patient attachable electrode signals, E_{LL}, E_{LA}, E_{RA}, E_{C1}, E_{C2}, even though the ECG lead signals derived from them are being generated accurately. A system according to invention principles addresses these limitations and associated problems.

### BRIEF SUMMARY OF THE INVENTION

In accordance with principles of the present invention, an electrode connectivity determination system is coupled to a plurality of patient attachable electrodes. Such a system includes a comparator for performing a comparison of a low frequency differential signal derived from at least two of the patient attachable electrodes with a predetermined threshold signal and a decision processor for generating a signal indicating an electrode signal is at least potentially degraded in response to said first comparison.

### BRIEF DESCRIPTION OF THE DRAWING

In the drawing:

FIG. 1 is a diagram partially in schematic form and partially in physical form illustrating the arrangement of patient attachable electrodes on a patient and a portion of electrical circuitry associated with the electrodes;

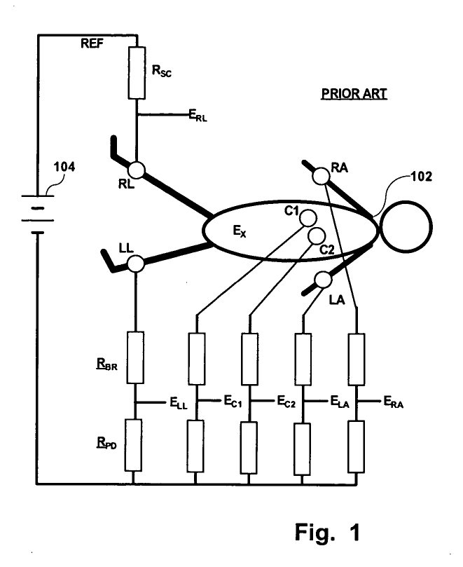

FIG. 2 is a schematic diagram of an equivalent circuit representing a portion of the electrode connectivity determination system illustrated in FIG. 1;

FIG. 3a, and FIG. 3b are block diagrams of a portion of the blanking processor illustrated in FIG. 2;

FIG. 4 is a block diagram of an arrangement of the system illustrated in FIG. 1, FIG. 2, FIG. 3a, and FIG. 3b in which a remote portion and a central monitor are linked via a communications channel; and

FIG. 5 is a flow chart illustrating the processing of patient attachable electrode signals to determine connectivity of electrodes on a patient.

### DETAILED DESCRIPTION OF THE INVENTION

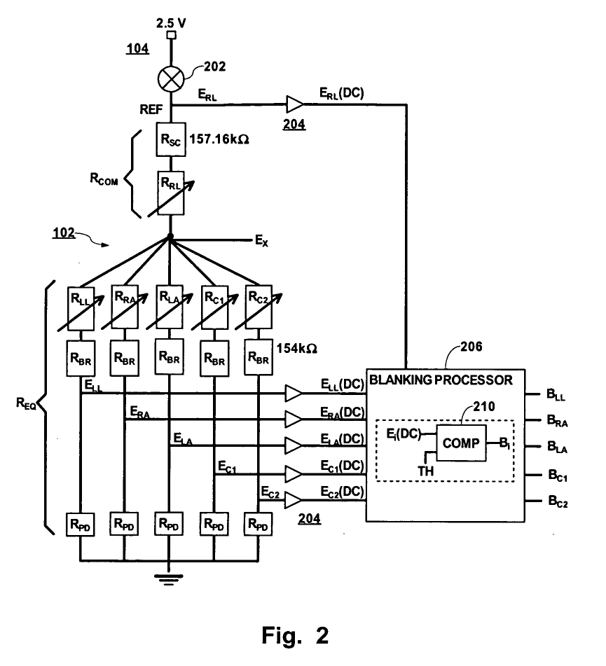

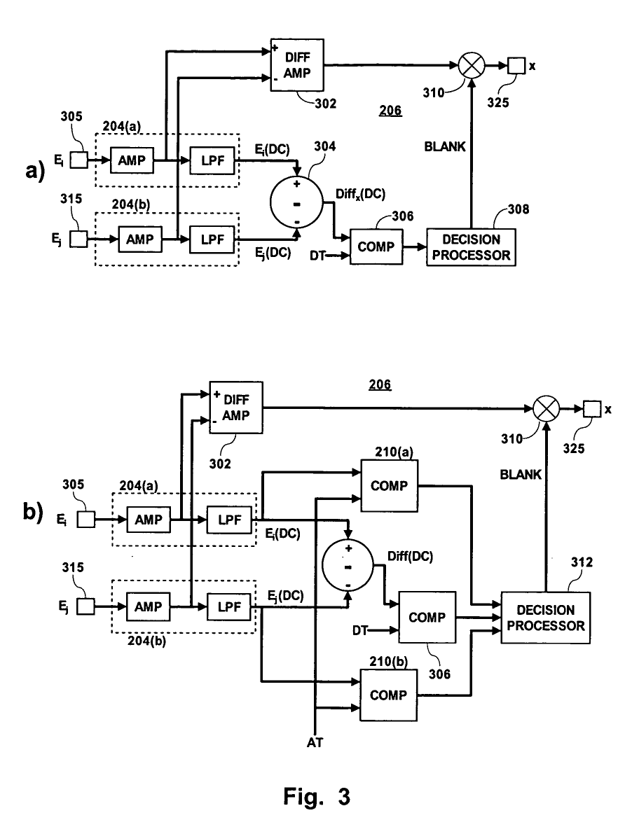

FIG. 3 is a block diagram of a portion of the blanking processor 206 illustrated in FIG. 2. In FIG. 3, those elements which are the same as those in FIG. 2 are designated by the same reference number and are not described in detail. In FIG. 3a, a first input terminal 305 and a second input terminal 315 are coupled to appropriate ones, Eᵢ and Eⱼ, of the plurality, E_{RL}, E_{LL}, E_{LA}, E_{RA}, E_{C1}, E_{C2}, of patient attachable electrode terminals (FIG. 2) as described below. The first input terminal 305 is coupled to an input terminal of a first amplifier AMP. The first amplifier AMP is a unity gain buffer amplifier and is coupled to a non-inverting input terminal of a differential amplifier 302 and an input terminal of a first LPF. The combination of the first buffer amplifier AMP and first low pass filter LPF form the first buffer amplifier and LPF 204(a) (of FIG. 2). The second input terminal 315 is coupled to an input terminal of a second amplifier AMP. The second amplifier AMP is also a unity gain buffer amplifier and is coupled to an inverting input terminal of the differential amplifier 302 and an input terminal of a second LPF. The combination of the second amplifier AMP and second low pass filter LPF form the second buffer amplifier and LPF 204(b). An output terminal of the differential amplifier 302 is coupled to a signal input terminal of a blanking circuit 310. An output terminal of the blanking circuit 310 is coupled to an output terminal 325. The output terminal 325 generates one, x, of the respective ECG lead signals, i.e. I, II, V, V+, etc., and is coupled to ECG lead signal processing and display circuitry (not shown). Respective output terminals of the first and second buffer amplifiers and LPFs 204(a) and 204(b) are coupled to subtrahend and minuend input terminals of a subtractor 304. A difference output terminal of the subtractor 304 is coupled to a signal input terminal of a comparator 306. A source of a differential threshold signal DT is coupled to a comparison input terminal of the comparator 306. An output terminal of the comparator 306 is coupled to an input terminal of a decision processor 308. An output terminal of the decision processor 308 is coupled to a control input terminal of the blanking circuit 310.

In operation, the differential amplifier 302 generates a differential signal representing an ECG lead signal, from the patient attachable electrode signals supplied to it. More specifically, in the illustrated embodiment, the I lead signal is calculated by coupling the E_{LA}patient attachable electrode terminal to the first input terminal 305 and the E_{RA} patient attachable electrode terminal of the second input terminal 315, thus subtracting the RA patient attachable electrode signal from the LA patient attachable electrode signal. Similarly, the II lead signal is calculated by coupling the E_{LL} patient attachable electrode terminal to the first input terminal 305 and coupling the E_{RA} patient attachable electrode terminal to the second input terminal 315, thus subtracting the RA patient attachable electrode signal from the LL patient attachable electrode signal.

One skilled in the art understands that calculation of other ECG lead signals requires calculation of intermediate signals and forming a differential signal in response to that intermediate signal. For example, an intermediate signal W is generated to be the average of the limb patient attachable electrode signals, E_{RA}, E_{LA}, and E_{LL}. That is

$$W = \frac{{E}_{LL} + {E}_{RA} + {E}_{LA}}{3} .$$

The V lead signal is calculated by generating the W signal as described above, coupling the E_{C1} patient attachable electrode terminal of the first input terminal 305 and W to the second input terminal, thus subtracting the W signals from the C1 patient attachable electrode signal. Similarly, the V+ lead is calculated by generating the W signal as described above, coupling the E_{C2} patient attachable electrode terminal of the first input terminal 305 and W to the second input terminal, thus subtracting the W signal from the C2 patient attachable electrode signal. One skilled in the art understands how to design and implement a circuit to properly calculate the required intermediate signal W.

One skilled in the art also understands that the differential amplifier 302 generates an output signal accurately provided that it is operated within known constraints. One such constraint is that the input signals must remain within the limits of the power supplied to the amplifier. That is, the input signal may not exceed the voltage of the power or ground levels. Typical differential amplifiers 302 also include a baseline recovery or autoblock circuit. This circuit removes a DC offset in the output signal from the differential amplifier. However, the baseline recovery or autoblock circuit can saturate and cease to operate accurately. To prevent saturation of the autoblock circuit, the difference between the DC levels of the respective input signals may not exceed a predetermined level. Other constraints exist and are understood by those skilled in the art.

The respective buffer amplifiers and LPFs 204(a) and 204(b) buffer and low pass filter the patient attachable electrode signals Eᵢ and Eⱼ. They produce low frequency or DC signals Eᵢ(DC) and Eⱼ(DC). These low frequency or DC signals are subtracted in the subtractor 304 to produce a low frequency or DC differential signal Diffₓ(DC). The subtraction corresponds to the processing performed by the differential amplifier 302. Thus, the signal Diffₓ(DC) is the low frequency or DC component of the ECG lead signal x produced by the differential amplifier 302.

The differential threshold signal DT is set to a level which approximates the level at which the autoblock function activates in the differential amplifier 302. If the output signal from the comparator 306 indicates that the differential threshold was exceeded, then the decision processor 308 generates a blanking signal, indicating that a patient attachable electrode signal is at least potentially degraded, which conditions the blanking circuit 310 to blank the x lead signal. If the output signal from the comparator 306 indicates that the differential threshold was not exceeded, then the decision processor 308 generates a blanking signal which conditions the blanking circuit 310 to pass the ECG lead signal x unblanked. As used herein, a decision processor receives signals representing the results of comparing the differential patient attachable electrode signals to the differential threshold signal DT, and possibly representing other comparisons, and processes those signals to decide whether the ECG lead signal represented by that differential signal is potentially degraded and should be blanked.

More specifically, in the illustrated embodiment, for the ECG lead signal I, the Eᵢ input terminal is coupled to the E_{LA} patient attachable electrode terminal and the Eⱼ input terminal is coupled to the E_{RA} patient attachable electrode terminal. The blanking processor 206 generates a low frequency differential signal DIFF_{I}(DC) corresponding to the ECG lead signal I, and representing the difference between the left arm patient attachable electrode signal E_{LA} and the right arm patient attachable electrode signal E_{RA}. This low frequency differential signal DIFF_{I}(DC) is compared to the differential threshold signal DT. If the differential threshold signal is exceeded, the signal indicating that the ECG lead signal I is potentially degraded is generated.

Similarly, for the ECG lead signal II, the Eᵢ input terminal is coupled to the E_{LL} patient attachable electrode terminal and the Eⱼ input terminal is coupled to the E_{RA} patient attachable electrode terminal. The blanking processor 206 generates a low frequency differential signal DIFF_{II}(DC) corresponding to the ECG lead signal II, and representing the difference between the left leg patient attachable electrode signal E_{LL} and the right arm patient attachable electrode signal E_{RA}. This low frequency differential signal DIFF_{II}(DC) is compared to the differential threshold signal DT. If the differential threshold signal is exceeded, the signal indicating that the ECG lead signal II is potentially degraded is generated.

For the ECG lead signal V, the Eᵢ input terminal is coupled to the E_{C1} patient attachable electrode terminal and the Eⱼ input terminal is coupled to receive the W signal. The blanking processor 206 also generates a low frequency differential signal DIFF_{V}(DC) corresponding to the ECG lead signal V, and representing the difference between the first chest patient attachable electrode signal E_{C1} and the W signal (i.e. the averaged signal derived from the left leg patient attachable electrode signal E_{LL}, the right arm patient attachable electrode signal E_{RA} and the left arm patient attachable electrode signal E_{LA}). This low frequency differential signal DIFF_{V}(DC) is compared to the differential threshold signal DT. If the differential threshold signal is exceeded, the signal indicating that the ECG lead signal V is potentially degraded is generated.

Similarly, for the ECG lead signal V+, the Eᵢ input terminal is coupled to the E_{C2} patient attachable electrode terminal and the Eⱼ input terminal is coupled to receive the W signal. The blanking processor 206 generates a low frequency differential signal DIFF_{V+}(DC) corresponding to the ECG lead signal V+, and representing the difference between the second chest patient attachable electrode signal E_{C2} and the W signal (i.e. the averaged signal derived from the left leg patient attachable electrode signal E_{LL}, the right arm patient attachable electrode signal E_{RA} and the left arm patient attachable electrode signal E_{LA}). This low frequency differential signal DIFF_{V+}(DC) is compared to the differential threshold signal DT. If the differential threshold signal is exceeded, the signal indicating that the ECG lead signal V+ is potentially degraded is generated.

FIG. 3b illustrates an alternative to the blanking processor 206 illustrated in FIG. 3a. Those elements in FIG. 3b which are the same as those illustrated in FIG. 2 and FIG. 3a are designated by the same reference numbers and are not described in detail below. In FIG. 3b, the output terminal of the first buffer amplifier and LPF 204(a) is further coupled to a signal input terminal of a second comparator 210(a). A comparison input terminal of the second comparator 210(a) is coupled to a source of a second, absolute, threshold signal AT. The output terminal of the second buffer amplifier and LPF 204(b) is further coupled to a signal input terminal of a third comparator 210(b). A comparison input terminal of the third comparator 210(b) is also coupled to the source of the absolute threshold signal AT. The respective output terminals of the first, second and third comparators 306, 210(a) and 210(b) are coupled to corresponding input terminals of a decision processor 312. An output terminal of the decision processor 312 is coupled to the control input terminal of the blanking circuit 310.

In operation, the blanking processor 206 illustrated in FIG. 3b operates similarly to that illustrated in FIG. 3a. The second and third comparators 210(a) and 210(b) operate as described above with respect to FIG. 2. The respective low frequency or DC signals Eᵢ(DC) and Eⱼ(DC) from buffer amplifier and LPFs 204(a) and 204(b) are compared to the absolute threshold AT in the second and third comparators 210(a) and 210(b). As described above, if electrical contact between a patient attachable electrode RL, LL, RA, LA, C1, C2 and the patient is degraded, the common branch patient attachable electrodes are pulled toward ground. The absolute threshold AT is set to detect this condition. The decision processor 312 receives the results of the comparisons from the first, second and third comparators, 306, 210(a) and 210(b) respectively, and generates a blanking signal conditioning the blanking circuit 310 to blank the ECG x lead signal if the comparisons indicate that a corresponding threshold was exceeded. Otherwise, the decision processor 312 generates a blanking signal conditioning the blanking circuit to pass the ECG x lead signal unchanged.

More specifically, in the illustrated embodiment, for the ECG lead I signal, the left arm patient attachable electrode signal E_{LA} is coupled to the first terminal Eᵢ and the right arm patient attachable electrode signal E_{RA} is coupled to the second terminal Eⱼ. The decision processor 312 generates a signal indicating that the ECG lead I signal is at least potentially degraded in response to at least one of: (a) the left arm patient attachable electrode signal E_{LA} exceeding the predetermined absolute threshold AT signal; (b) the right arm patient attachable electrode signal E_{RA} exceeding the predetermined absolute threshold AT signal; and (c) the differential signal Diff_{I}(DC) exceeding the predetermined differential threshold DT signal (as described above with respect to FIG. 3a). Similarly, for the ECG lead II signal, the left leg patient attachable electrode signal E_{LL} is coupled to the first terminal Eᵢ and the right arm patient attachable electrode signal E_{RA} is coupled to the second terminal Eⱼ. The decision processor 312 generates a signal indicating that the ECG lead II signal is at least potentially degraded in response to at least one of: (a) the left leg patient attachable electrode signal E_{LL} exceeding the predetermined absolute threshold AT signal; (b) the right arm patient attachable electrode signal E_{RA} exceeding the predetermined absolute threshold AT signal; and (c) the differential signal Diff_{II}(DC) exceeding the predetermined differential threshold DT signal.

As described above, the ECG V and V+ chest lead signals are generated in response to the W signal, representing an averaged signal derived from Left Leg, Right Arm and Left Arm electrode signals E_{LL}, E_{RA}, E_{LA}. For the ECG lead V signal, the first chest patient attachable electrode signal E_{C1} is coupled to the first terminal Eᵢ and the W signal is coupled to the second terminal Eⱼ. The decision processor 312 generates a signal indicating that the ECG lead V signal is at least potentially degraded in response to at least one of: (a) the first chest patient attachable electrode signal E_{C1} exceeding the predetermined absolute threshold signal; (b) the averaged W signal exceeding the predetermined absolute threshold signal; and (c) the differential signal Diff_{V}(DC) exceeding the predetermined differential threshold signal. For the ECG lead V+ signal, the second chest patient attachable electrode signal E_{C2} is coupled to the first terminal Eᵢ and the W signal is coupled to the second terminal Eⱼ. The decision processor 312 generates a signal indicating that the ECG lead V+ signal is at least potentially degraded in response to at least one of: (a) the second chest patient attachable electrode signal E_{C2} exceeding the predetermined absolute threshold AT signal; (b) the averaged W signal exceeding the predetermined absolute threshold AT signal; and (c) the differential signal Diff_{V}+(DC) exceeding the predetermined differential threshold DT signal.

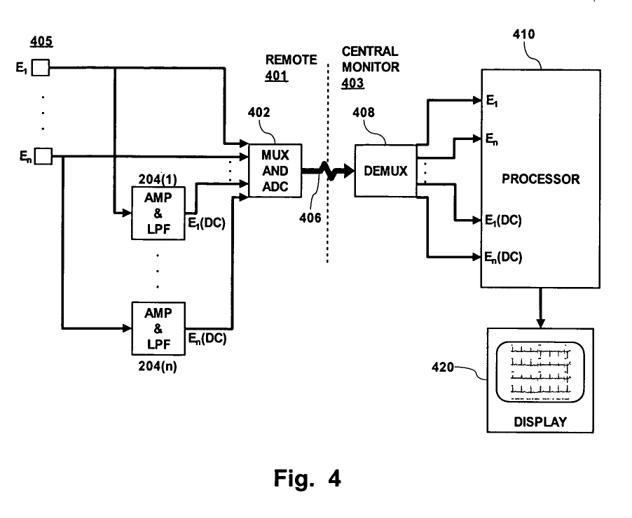

One skilled in the art understands that ECG monitoring systems often include a remote portion, for connecting patient attachable electrodes to a patient; and a central monitor, for receiving ECG signals from the remote portion, processing the received signals and displaying the resulting ECG lead signals on an image display device. FIG. 4 is a block diagram of a portion of a blanking processor 206 illustrated in FIG. 3a and FIG. 3b. In FIG. 4, those elements which are the same as those illustrated in FIG. 2 or FIG. 3 are designated by the same reference number and are not described in detail. In FIG. 4, a remote portion 401 is coupled to a central monitor 403 via a communications channel 406. In the illustrated embodiment, a plurality 405 of input terminals E₁ to Eₙ are coupled to respective patient attachable electrode terminals, E_{RL}, E_{LL}, E_{LA}, E_{RA}, E_{C1}, E_{C2}. The plurality 405 of input terminals E₁ through Eₙ are coupled to respective input terminals of a multiplexer (MUX) and analog-to-digital converter (ADC) 402 and to respective input terminals of respective buffer amplifiers and LPFs 204(1) through 204(n). Respective output terminals of the buffer amplifiers and LPFs 204(1) through 204(n) are coupled to corresponding further input terminals of the MUX and ADC 402. A digital output terminal of the MUX and ADC 402 is coupled through the communications channel 406 to a demultiplexer DEMUX 408 in the central monitor 403. Respective output terminals of the DEMUX 408 produce digital signals representing the multiplexed signals E₁ through Eₙ and E₁(DC) through Eₙ(DC) and are coupled to corresponding input terminals of a processor 410. An output terminal of the processor 410 is coupled to an input terminal of a display device 420.

In operation, the MUX portion of 402 couples one of the plurality 405 of patient attachable electrode signals E₁ through Eₙ or of the plurality of buffered and LPFed signals E₁(DC) through Eₙ(DC) input signal at a time to the ADC portion of 402. The ADC portion of 402 samples and converts that analog signal to a multibit digital electrical signal. These samples form a time division multiplexed multibit digital sample stream. This multibit digital sample stream is transmitted to the central monitor 403 via the communications channel 406.

The communications channel 406 may be implemented as a parallel digital data bus or a serial data connection. The digital samples may be accompanied by one or more clock signals to synchronize the operation of the central monitor 403 and the remote 401 or may be self clocking. The digital samples may also be transmitted in packetized form. The communications channel 406 may be a wired connection or a wireless connection. More specifically, in the illustrated embodiment, the communications channel may be an Ethernet or any other appropriate wired connection or a Wi-Fi or Bluetooth or any other appropriate wireless connection. The packets may be formatted according to TCP/IP formatting or any other appropriate packet protocol. One skilled in the art understands that a minimum sample rate must be maintained in order to provide the bandwidth necessary to process and display ECG lead signals derived from the patient attachable electrode signals E₁ through Eₙ. One skilled in the art also understands that the sample rate for the low frequency or DC patient attachable electrode signals E₁(DC) through Eₙ(DC) may be substantially lower than the sampling rate for the patient attachable electrode signals E₁ through Eₙ.

The DEMUX 408 extracts the digital data signals from the communications channel 406 and generates respective separate digital data streams for each patient attachable electrode signal E₁ through Eₙ and for each low frequency or DC patient attachable electrode signal E₁(DC) through Eₙ(DC). These data streams are processed by the processor 410.

As used herein, a processor operates under the control of an executable application to (a) receive information from an input information device, (b) process the information by manipulating, analyzing, modifying, converting and/or transmitting the information, and/or (c) route the information to an output information device. A processor may use, or comprise the capabilities of, a controller or microprocessor, for example. The processor may operate with a display processor or generator. A display processor or generator is a known element for generating signals representing display images or portions thereof. A processor and a display processor comprises any combination of, hardware, firmware, and/or software.

An executable application as used herein comprises code or machine readable instructions for conditioning the processor to implement predetermined functions, such as those of an operating system, patient monitoring and/or treatment system or other information processing system, for example, in response user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters.

The processor 410 executes an executable procedure for receiving the respective digital data streams. The patient attachable electrode digital signals E₁ through Eₙ are processed, as described above, to generate ECG lead signals. The low frequency or DC patient attachable electrode signals E₁(DC) through Eₙ(DC) are also processed as illustrated in FIG. 3 and described in more detail above to generate blanking signals for the ECG lead signals.

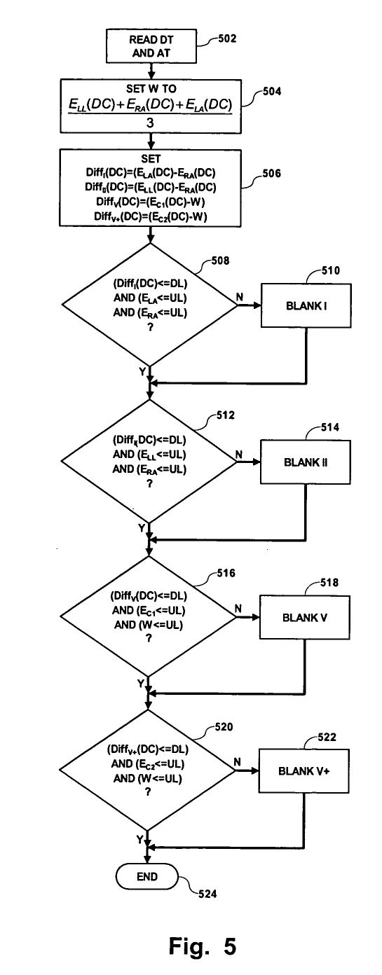

FIG. 5 is a flow chart illustrating the processing of patient attachable electrode signals performed by the processor 410 (FIG. 4) to determine connectivity of electrodes on a patient. In step 502 the differential threshold DT data and the absolute threshold AT data are read from storage. In step 504, the variable W is set to the average of the E_{LL}(DC), E_{RA}(DC) and E_{LA}(DC) signals. In step 506 respective differential signals Diff_{I}(DC), Diff_{II}(DC), Diff_{V}(DC) and Diff_{V+}(DC) are calculated for the ECG lead signals I, II, V and V+. he differential signal Diff_{I}(DC) is compared to the differential threshold DT signal and the component patient attachable electrode signals E_{LA}(DC) and E_{RA}(DC) are compared to the absolute threshold signals AT. If the comparisons indicate that the signals are within the thresholds, the next ECG lead signal is checked in step 512. Otherwise the ECG lead signal I is blanked in step 510. In step 512, blanking for the ECG lead II signal is checked. The differential signal Diff_{II}(DC) is compared to the differential threshold DT signal and the component patient attachable electrode signals E_{LL}(DC) and E_{RA}(DC) are compared to the absolute threshold signals AT. If the comparisons indicate that the signals are within the thresholds, the next ECG lead signal is checked in step 516. Otherwise the ECG lead signal II is blanked in step 514. In step 516, blanking for the ECG lead V signal is checked. The differential signal Diff_{V}(DC) is compared to the differential threshold DT signal and the component patient attachable electrode signal E_{C1}(DC) and the W signal are compared to the absolute threshold signals AT. If the comparisons indicate that the signals are within the thresholds, the next ECG lead signal is checked in step 520. Otherwise the ECG lead signal V is blanked in step 518. In step 520, blanking for the ECG lead V+ signal is checked. The differential signal Diff_{V+}(DC) is compared to the differential threshold DT signal and the component patient attachable electrode signal E_{C2}(DC) and the W signal are compared to the absolute threshold signals AT. If the comparisons indicate that the signals are within the thresholds, the executable procedure ends in step 524. Otherwise the ECG lead signal V+ is blanked in step 522.

One skilled in the art understands that in step 506, to simplify comparisons to thresholds, the absolute value of the differential signals Diff_{I}(DC), Diff_{II}(DC), Diff_{V}(DC) and Diff_{V+}(DC) may also be calculated. In steps 508, 512, 516 and 520, the absolute values of the differential signals Diff_{I}(DC), Diff_{II}(DC), Diff_{V}(DC) and Diff_{V+}(DC) may be compared to the appropriate thresholds.

The processor 410 then conditions a display generator to produce an image representative signal displaying the unblanked ECG lead signals and not displaying blanked ECG lead signals on the display device 420. One skilled in the art understands that additional displays may be generated and displayed for the user, such as visual indicators of which patient attachable electrode signals exceed the threshold and/or other parameters. Non-visual signals may also be generated such as audible alarms.

Setting of the absolute threshold AT and the differential threshold DT involves determining the constraints of the front end circuits, i.e. common mode voltage, DC differential voltage, rail voltages, etc., and analyzing the equivalent circuit of FIG. 2 to determine the patient attachable electrode resistances R_{RL}, R_{RA,}R_{LA}, R_{LL}, R_{C1}, R_{C2}, at which the constraints are violated. The absolute threshold AT signals and the differential threshold DT signals are set based on this analysis.

For example, in order to maintain the input signals of the differential amplifiers within the power rails, the DC patient attachable electrode signal must not become too low or too high in voltage. However, referring to FIG. 2, the equivalent circuit is very complex with six unknown resistances (the patient attachable electrode resistances R_{RL}, R_{RA}, R_{LA}, R_{LL}, R_{C1}, R_{C2}). A simple analytical solution to detecting an patient attachable electrode signal near a power rail is not available. In order to simplify the search for an absolute threshold AT signal which maintains operation within this constraint, three simplifications may be used. First, the patient attachable electrode resistances may be concurrently changed, i.e. increased equally, simulating a gradual drying of the patient attachable electrodes RL, LL, RA, LA, C1, C2. Second, the resistance of the reference, common branch, patient attachable electrode RL may be changed to simulate impaired electrical contact between the RL patient attachable electrode and the patient body. Third, the resistance of one of the non-common branch patient attachable electrodes LL, RA, LA, C1, C2 may be changed to simulate impaired electrical contact between the non-common branch electrode LL, RA, LA, C1, C2 and the patient body.

The equivalent circuit of FIG. 2 may be simulated to determine the respective patient attachable electrode signals E_{RL}, E_{RA}, E_{LA}, E_{LL}, E_{C1}, E_{C2} resulting as changes are made to the patient attachable electrode resistances R_{RL}, R_{RA}, R_{LA}, R_{LL}, R_{C1}, R_{C2}, in the manner described above. Equations (1) through (5), below, are derived from Ohm's law and describe the DC operation of the circuit illustrated in FIG. 2. The patient attachable electrode resistances R_{RL}, R_{RA}, R_{LA}, R_{LL}, R_{C1}, R₂ are initially set to zero ohms, and varied as described above. Equations (1) through (5) are solved to determine the resulting patient attachable electrode signals E_{RL}, E_{RA}, E_{LA}, E_{LL}, E_{C1}, E_{C2}. When a patient attachable electrode signal E_{RL}, E_{RA}, E_{LA}, E_{LL}, E_{C1}, E_{C2} exceeds the power rail input constraint for the differential amplifier, the absolute threshold AT is set to a value to blank the ECG lead signals associated with that patient attachable electrode signal E_{RL}, E_{RA}, E_{LA}, E_{LL}, E_{C1}, E_{C2} before that constraint is exceeded.
R_{COM}=R_{SC}+R_{RL} where R_{SC}=157.16KΩ (1)
R_{EQ}=R_{RA}+R_{BR}+R_{PD}∥R_{LA}+R_{BR}+R_{PD}∥R_{LL}+R_{BR}+R_{PD}∥R_{C1}+R_{BR}+R_{PD}∥R_{C2}+R_{BR}+R_{PD} (2)

$$\begin{matrix}{R}_{EQ} = \frac{1}{\frac{1}{{R}_{RA} + {R}_{BR} + {R}_{PD}} + \frac{1}{{R}_{LA} + {R}_{BR} + {R}_{PD}} + \frac{1}{{R}_{LL} + {R}_{BR} + {R}_{PD}} + \frac{1}{{R}_{C 1} + {R}_{BR} + {R}_{PD}} + \frac{1}{{R}_{C 2} + {R}_{BR} + {R}_{PD}}} & ( 3 ) \\ {E}_{X} = REF \frac{{R}_{EQ}}{{R}_{COM} + {R}_{EQ}} & ( 4 ) \\ {E}_{i} = {E}_{X} \frac{{R}_{PD}}{{R}_{PD} + {R}_{i}} ( for i = RA , LA , LL , C1 , C2 ) & ( 5 )\end{matrix}$$

The maximum DC differential voltage input constraint on the differential amplifier may be simulated in a similar manner. In this case, as the patient attachable electrode resistances R_{RL}, R_{RA}, R_{LA}, R_{LL}, R_{C1}, R_{C2} are changed in the manner described above, the circuit of FIG. 2 is simulated, using equations (1) through (5). Differential signals between patient attachable electrode signals E_{RL}, E_{RA}, E_{LA}, E_{LL}, E_{C1}, E_{C2} appropriate for calculating the ECG lead signals (i.e. E_{LA}(DC)-E_{RA}(DC), E_{LL}(DC)-E_{RA}(DC), E_{C1}(DC)-W, E_{C2}(DC)-W) are calculated. When a differential signal exceeds the maximum DC differential voltage input constraint for the differential amplifier, the differential threshold DT is set to a value to blank the ECG lead signals associated with that differential signal before that constraint is exceeded.

It is further possible for the absolute threshold AT and differential threshold DT signals to be adaptively adjusted. For example, referring again to FIG. 4, the processor 410 makes the comparisons set out in FIG. 5. The processor 410 may further adjust the absolute threshold AT signals based on the results of the comparisons of the differential patient attachable electrode signals to the differential threshold DT signal. In this manner, the processor 410 may adaptively adjust the absolute threshold AT signal so that it does not result in unnecessarily blanking an ECG lead signal.

The present invention has been described by reference to an ECG patient monitoring system using six patient attachable electrodes to generate six ECG lead signals. However, one skilled in the art understands that the number of patient attachable electrodes and ECG leads is not germane. That is, an ECG patient monitor displaying 12 ECG lead signals using 10 patient attachable electrodes may also use the invention described above. Further, an ECG patient monitor displaying a subset of the ECG lead signals using a subset of patient attachable electrodes, such as a three ECG lead signal system, or an ECG patient monitor using a superset of ECG lead signals using additional patient attachable electrodes to provide additional views of the patient cardiac function, may also use the invention described above.

The steps illustrated in FIG. 5 and the functions provided by the circuitry illustrated in FIG. 3 and FIG. 4 may be performed by hardware specially designed and implemented to provide those functions, or by software, firmware software, or any combination of these.

Table 1 describes the variables and constants used above in describing the present invention.

TABLE 1
Variables/Constants

| | |
|---|---|
| R_{COM} | Total Resistance in the common branch |
| R_{EQ} | Equivalent resistance of the non-common branches |
| REF | Voltage driving the common lead branch |
| E_{X} | Voltage appearing at divide point between common |
| | and non-common branches |
| R_{PD} | Pull down resistance in non-common branches |
| R_{RA} | Right Arm patient attachable electrode resistance |
| R_{LA} | Left Arm patient attachable electrode resistance |
| R_{LL} | Left Leg patient attachable electrode resistance |
| R_{C1} | First Chest patient attachable electrode resistance |
| R_{C2} | Second Chest patient attachable electrode resistance |
| R_{BR} | Series non-common branch resistance. |
| R_{SC} | Series common branch resistance. |
| DT | Differential Threshold |
| AT | Absolute Threshold |
| E_{RA} | Right Arm patient attachable electrode voltage |
| E_{LA} | Left Arm patient attachable electrode voltage |
| E_{LL} | Left Leg patient attachable electrode voltage |
| E_{C1} | First Chest patient attachable electrode voltage |
| E_{C2} | Second Chest patient attachable electrode voltage |

What is claimed is:

1. An electrode connectivity determination system, coupleable to a plurality of patient attachable electrodes, comprising:
a first comparator for performing a first comparison of a low frequency differential signal derived from at least two of said patient attachable electrodes with a predetermined threshold signal; and
a decision processor for generating a signal indicating an electrode signal is at least potentially degraded in response to said first comparison.

2. The system of claim 1 further comprising:
a second comparator for performing a second comparison of a low frequency signal derived from one of said patient attachable electrodes with second predetermined threshold signal; wherein:
said decision processor generates a signal indicating an electrode signal is at least potentially degraded in response to said first and said second comparisons

3. The system of claim 2 wherein said low frequency differential signal and said low frequency signal are respective substantially DC signals.

4. The system of claim 2 wherein said low frequency signal is derived by buffering and low pass filtering an electrical signal from said patient attachable electrode.

5. The system of claim 2 wherein said low frequency differential signal is derived by buffering and low pass filtering electrical signals from said at least two patient attachable electrodes.

6. The system of claim 5 wherein said low frequency differential signal is derived by analog to digital conversion of said buffered and low pass filtered electrical signals.

7. The system of claim 1 comprised in an ECG monitor wherein said decision processor generates a signal indicating an electrode signal corresponding to ECG lead signal I is at least potentially degraded in response to:
a low frequency differential signal, derived from data representing a difference between Left Arm and Right Arm patient attachable electrode signals, exceeding said first predetermined threshold signal; and
at least one of, (a) a Left Arm patient attachable electrode signal exceeding said second predetermined threshold signal, and (b) a Right Arm patient attachable electrode signal exceeding said second predetermined threshold signal.

8. The system of claim 1 comprised in an ECG monitor wherein said decision processor generates a signal indicating an electrode signal corresponding to ECG lead signal II is at least potentially degraded in response to:
a low frequency differential signal, derived from data representing a difference between Left Leg and Right Arm patient attachable electrode signals, exceeding said first predetermined threshold signal; and
at least one of, (a) a Left Leg patient attachable electrode signal exceeding said second predetermined threshold signal, and (b) a Right Arm patient attachable electrode signal exceeding said second predetermined threshold signal.

9. The system of claim 1 comprised in an ECG monitor wherein said decision processor generates a signal indicating an electrode signal corresponding to a first ECG chest lead signal V is at least potentially degraded in response to:
a low frequency differential signal, derived from data representing a difference between a First Chest patient attachable electrode signal and an averaged signal derived from Left Leg, Right Arm and Left Arm patient attachable electrode signals, exceeding said first predetermined threshold signal; and
at least one of, (a) the First Chest patient attachable electrode signal exceeding said second predetermined threshold signal and (b) said averaged signal exceeding said second predetermined threshold signal.

10. The system of claim 1 comprised in an ECG monitor wherein said decision processor generates a signal indicating an electrode signal corresponding to a second ECG chest lead signal V+ is at least potentially degraded in response to:
a low frequency differential signal, derived from data representing a difference between said Second Chest patient attachable electrode signal and an averaged signal, derived from Left Leg, Right Arm and Left Arm patient attachable electrode signals, exceeding said first predetermined threshold signal; and
at least one of, (a) the Second Chest patient attachable electrode signal exceeding said second predetermined threshold signal and (b) said averaged signal exceeding said second predetermined threshold signal.

11. The system of claim 1 wherein a patient attachable electrode signal is at least potentially degraded if said patient attachable electrode signal is affected by impaired electrical contact between the patient attachable electrode and a patient body.

12. The system of claim 11 wherein said impaired electrical contact occurs if at least one of, (a) said patient attachable electrode is detached from said patient body and (b) electrical resistivity between said patient attachable electrode and said patient body is significantly increased because of degraded electrical contact.

13. The system of claim 1 wherein said patient attachable electrodes are of a conventional 12 lead ECG signal set.

14. The system of claim 1 wherein said patient attachable electrodes are of a subset or reduced number of leads of a conventional 12 lead ECG signal set.

15. The system of claim 1 wherein said decision processor automatically adaptively adjusts said second predetermined threshold signal in response to said first comparison.

16. An electrode connectivity determination system, coupleable to a plurality of patient attachable electrodes, comprising:
a first comparator for performing a first comparison of a low frequency differential signal derived from at least two patient attachable electrodes with a first predetermined threshold signal;
a second comparator for performing a second comparison of a low frequency signal derived from a patient attachable electrode with a second predetermined threshold signal; and
a decision processor for generating a signal indicating an electrode signal is at least potentially degraded in response to said first and said second comparisons.

17. A system, coupleable to a plurality of patient attachable electrodes, for use in determining electrode connectivity, comprising:
a first comparator for performing a first comparison of a low frequency differential signal derived from at least two patient attachable electrodes with a first predetermined threshold signal;
a second comparator for performing a second comparison of a low frequency signal derived from a patient attachable electrode with a second predetermined threshold signal; and
a decision processor for automatically adaptively adjusting said second predetermined threshold signal in response to said first comparison.

18. A method for determining connectivity status of a plurality of patient attachable electrodes, comprising:
performing a first comparison of a low frequency differential signal derived from at least two patient attachable electrodes with a first predetermined threshold signal;
performing a second comparison of a low frequency signal derived from a patient attachable electrode with a second predetermined threshold signal; and
generating a signal indicating an electrode signal is at least potentially degraded in response to said first and said second comparison.
